(19) United States
(12) Patent Application Publication
(10) Pub. No.: US 2015/0099668 A1
(43) Pub. Date: Apr. 9, 2015

(54) METABOLITE BIOMARKERS FOR STAGING COLORECTAL CANCER

(71) Applicant: UTI LIMITED PARTNERSHIP, Calgary (CA)

(72) Inventors: Farshad Farshidfar, Calgary (CA); Aalim M. Weljie, Calgary (CA); Karen Kopciuk, Calgary (CA); Hans J. Vogel, Calgary (CA); Oliver F. Bathe, Calgary (CA)

(73) Assignee: UTI LIMITED PARTNERSHIP, Calgary, AB (CA)

(21) Appl. No.: 14/401,286

(22) PCT Filed: May 14, 2013
(86) PCT No.: PCT/IB2013/001532

§ 371 Date: Nov. 14, 2014

Related U.S. Application Data
(60) Provisional application No. 61/646,605, filed on May 14, 2012.

(51) Int. Cl. G01N 33/49; G01N 24/08; G01N 33/487

(52) U.S. Cl. CPC G01N 33/492; G01N 33/48707; G01N 24/08; H01J 49/0422
USPC 506/12; 506/15; 506/7; 506/19; 250/282; 324/309

(57) ABSTRACT

This document provides methods and materials for assessing metabolites in a sample from a mammal (e.g., human) for determining the nature and extent of colorectal cancer (CRC) metastasis. For example, the document relates to the diagnosis, staging and prognosis of CRC in a mammal.

This application claims benefit of priority to U.S. Provisional Application Ser. No. 61/646,605, filed May 14, 2012, the entire contents of which are hereby incorporated by reference.

### BACKGROUND

1. Field of the Invention

This document relates to methods and materials for assessing metabolite biomarker useful in determining colorectal cancer metastasis. In particular, the methods comprise the use of GC-MS and ¹H-NMR to assess serum metabolites characteristic of either liver-only metastasis as opposed to loco-regional or extrahepatic disease.

2. Background of the Invention

While most individuals with metastatic colorectal cancer (CRC) receive treatments with palliative intent, there are some who may benefit from more aggressive surgical therapy, with curative intent. The prototypical situation in which cure can still be achieved in the face of metastatic disease is when metastases are isolated to the liver. In patients with limited intrahepatic disease, and in the absence of extrahepatic disease, resection can result in a median survival of 40-58 months and a 5-year survival of 40-58% (Pawlik et al., 2005; Bathe et al., 2009; Pawlik et al., 2008; Shah et al., 2007). Presently, only 25-30% of patients with colorectal liver metastases have resectable disease. It is possible that earlier identification of the presence of liver metastases could increase the proportion of patients who could undergo surgery with curative intent. Therefore, biomarkers that facilitate early detection of liver-only metastases could be useful. In addition, biomarkers that reveal the presence of radiographically occult extra-hepatic disease could help to better select patients who would benefit from resection of liver metastases.

Recent biomarker discovery efforts have focused largely on the genome, the transcriptome and the proteome, using technologies that enable quantification of multiple biomolecules at once. In metabolomics, the biomarkers of interest consist of metabolites, small molecules which are intermediates, and products of metabolism, including molecules associated with energy storage and utilization; precursors to proteins and carbohydrates; regulators of gene expression; and signaling molecules. Thus, like the proteome, the metabolome represents a functional portrait of the cell or the organism. One potential advantage of metabolomics over proteomics is that metabolic changes may be more closely related to the immediate (patho)physiologic state of the individual. However, relatively few biomarker discovery efforts have focused on the metabolome to date, including those relating to CRC.

### SUMMARY OF THE INVENTION

In accordance with the present invention, there is provided a method of characterizing metastatic colorectal cancer comprising (a) obtaining a mammalian biological sample; (b) analyzing the biological sample from the mammal with gas chromatography-mass spectrometry to determine the level(s) of one or more core biomarkers set forth in Table 2B and Table 2D; and (c) comparing the level(s) of the one or more core biomarkers in the sample to metastatic and/or normal reference levels of the one or more core biomarkers in order to assess the characterize metastatic disease in the mammal. Step (b) may comprise analyzing the levels of two, three, four, five, six, seven, eight, nine, ten, eleven, twelve, thirteen, fourteen, fifteen, sixteen, seventeen, eighteen, nineteen, twenty, twenty-five, thirty, thirty-five, forty, fifty or all of the core biomarkers in Tables 2B and 2D. One or more optional biomarkers from the tables may be analyzed as well.

The biological sample is a body fluid, such as a urine sample, a blood sample, a serum sample or a plasma sample. Analyzing may comprise determining one or more core markers in Table 2B, or all the core markers in Table 2B. Analyzing may comprise determining one or more core markers in Table 2D, or all the markers in Table 2D. The mammal may be a human. Characterizing may distinguish liver-only metastasis from locoregional colorectal or extrahepatic metastatic disease.

In another embodiment, there is provided a kit for characterizing metastatic colorectal cancer in a mammal, the kit comprising reagents suitable for determining levels of a plurality of core biomarkers in a test sample using gas chromatography-mass spectrometry, wherein the plurality of core biomarkers comprises two or more of the core biomarkers in Table 2B and Table 2D. The kit may further comprise one or more control samples comprising predetermined levels of the two or more core biomarkers. The kit may further comprise reagents suitable for determining the levels of two, three, four, five, six, seven, eight, nine, ten, eleven, twelve, thirteen, fourteen, fifteen, sixteen, seventeen, eighteen, nineteen, twenty, twenty-five, thirty, thirty-five, forty, fifty or all of the core biomarkers in Table 2B and Table 2D. The reagents may serve to distinguish liver-only colorectal metastasis from locoregional colorectal cancer. The reagents may alternatively serve to distinguish liver-only colorectal metastasis from extrahepatic metastatic disease. Reagents for analyzing one or more optional biomarkers from the tables may be included as well.

In yet another embodiment, there is provided a method of staging metastatic colorectal cancer comprising (a) obtaining a mammalian biological sample; (b) analyzing the biological sample from the mammal using gas chromatograph-mass spectrometry to determine the level(s) of one or more core biomarkers set forth in Table 2B and Table 2D; and (c) comparing the level(s) of the one or more biomarkers in the sample to metastatic and/or normal reference levels of the one or more core biomarkers in order to stage disease in the mammal. One or more optional biomarkers from the tables may be analyzed as well.

Still a further embodiment, there is provided a method of determining an appropriate therapy for colorectal cancer comprising (a) obtaining a mammalian biological sample; (b) analyzing the biological sample from the mammal using gas chromatograph-mass spectrometry to determine the level(s) of one or more core biomarkers set forth in Table 2B and Table 2D; and (c) comparing the level(s) of the one or more core biomarkers in the sample to metastatic and/or normal reference levels of the one or more core biomarkers in order to assess the extent of metastatic disease in the mammal, thereby indicating the appropriate type of therapy. The method may further comprise treating said mammal. The treatment may be surgery when the mammal exhibits biomarkers indicative of liver-only colorectal metastasis. The treatment may be chemotherapy when the mammal exhibits biomarkers indicative of extrahepatic metastatic disease. One or more optional biomarkers from the tables may be analyzed as well.

In an additional embodiment, there is provided a method of characterizing metastatic colorectal cancer comprising (a) obtaining a mammalian biological sample; (b) analyzing the biological sample from the mammal with ¹H-NMR spectroscopy to determine the level(s) of one or more core biomarkers set forth in Table 2A and Table 2C; and (c) comparing the level(s) of the one or more core biomarkers in the sample to metastatic and/or normal reference levels of the one or more core biomarkers in order to assess the characterize metastatic disease in the mammal. Step (b) may comprise analyzing the levels of two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six or all of the core biomarkers in Table 2A and Table 2C. The biological sample may be a body fluid, such as a urine sample, a blood sample, a serum sample or a plasma sample. Analyzing may comprise determining one or more core markers in Table 2A, or determining all the core markers in Table 2A. Analyzing may comprise determining one or more core markers in Table 2C, or determining all the core markers in Table 2C. The mammal may be a human. Characterizing distinguishes liver-only colorectal metastasis from locoregional colorectal metastasis or extrahepatic metastatic disease. One or more optional biomarkers from the tables may be analyzed as well.

Another embodiment comprises a kit for characterizing metastatic colorectal cancer in a mammal, the kit comprising reagents suitable for determining levels of a plurality of core biomarkers in a test sample using ¹H-NMR spectroscopy, wherein the plurality of core biomarkers comprises two or more of the core biomarkers in Table 2A and Table 2C. The kit may further comprise one or more control samples comprising predetermined levels of the two or more core biomarkers. The kit may further comprise reagents suitable for determining the levels of two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six or all of the core biomarkers in Table 2A and Table 2C. The reagents may distinguish liver-only colorectal metastasis from locoregional colorectal cancer. Alternatively, the reagents may distinguish liver-only colorectal metastasis from extrahepatic metastatic disease. Reagents for analyzing one or more optional biomarkers from the tables may be included as well.

An additional embodiment comprises a method of staging metastatic colorectal cancer comprising (a) obtaining a mammalian biological sample; (b) analyzing the biological sample from the mammal using ¹H-NMR spectroscopy to determine the level(s) of one or more core biomarkers set forth in Table 2A and Table 2C; and (c) comparing the level(s) of the one or more core biomarkers in the sample to metastatic and/or normal reference levels of the one or more core biomarkers in order to stage disease in the mammal. One or more optional biomarkers from the tables may be analyzed as well.

Yet an additional embodiment comprises a method of determining an appropriate therapy for colorectal cancer comprising (a) obtaining a mammalian biological sample; (b) analyzing the biological sample from the mammal using ¹H-NMR spectroscopy to determine the level(s) of one or more core biomarkers set forth in Table 2A and Table 2C; and (c) comparing the level(s) of the one or more core biomarkers in the sample to metastatic and/or normal reference levels of the one or more core biomarkers in order to assess the extent of metastatic disease in the mammal, thereby indicating the appropriate type of therapy. The method may further comprise treating said mammal. The treatment may surgery when the mammal exhibits biomarkers indicative of liver-only colorectal metastasis or locoregional disease. The treatment may be chemotherapy when the mammal exhibits biomarkers indicative of extrahepatic metastatic disease. One or more optional biomarkers from the tables may be analyzed as well.

Also provided is a method and kit for early detection of liver metastases in patients with colorectal cancer using any of the methods, samples, and markers set forth above.

It is contemplated that any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.” “About” means plus or minus 5% of the stated value.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. Comparison of metabolomic profiles from patients with locoregional CRC and liver-only disease. (FIG. 1A) O-PLS-DA scatter plot depicting metabolomic profiles analyzed by ¹H NMR spectroscopy. (FIG. 1B) O-PLS-DA scatter plot depicting metabolomic profiles analyzed by GC-MS. (FIG. 1C) Coefficient plot demonstrating relative abundance of specific metabolites detected by ¹H NMR spectroscopy. Metabolites on the left are more abundant in sera from patients with liver metastases, and metabolites on the right are most abundant in locoregional disease. (FIG. 1D) Coefficient plot demonstrating relative abundance of specific metabolites detected by GC-MS. Only identified metabolites are included.

FIGS. 2A-D. Comparison of metabolomic profiles from patients with liver-only metastases and with extrahepatic metastases. (FIG. 2A) O-PLS-DA scatter plot depicting metabolomic profiles analyzed by ¹H NMR spectroscopy. (FIG. 2B) O-PLS-DA scatter plot depicting metabolomic profiles analyzed by GC-MS. (FIG. 2C) Coefficient plot demonstrating relative abundance of specific metabolites detected by ¹H NMR spectroscopy. Metabolites on the left are more abundant in extrahepatic metastases, and metabolites on the right are most abundant in liver metastases. (FIG. 2D) Coefficient plot demonstrating relative abundance of specific metabolites detected by GC-MS. Only identified metabolites are included.

FIGS. 3A-D. ROC curves depicting the predictive performance of generated classifiers in each comparison. (FIG. 3A) ROC curve illustrating performance of the NMR model in distinguishing liver-only metastases from locoregional CRC. (FIG. 3B) ROC curve illustrating performance of the GC-MS model in distinguishing liver-only metastases from locoregional CRC. (FIG. 3C) ROC curve for the NMR model distinguishing extrahepatic metastases from liver-only metastases. (FIG. 3D) ROC curve for the GC-MS model distinguishing extrahepatic metastases from liver-only metastases. TPF=true positive fraction, FPF=False positive fraction, AUC=area under the ROC curve.

FIGS. 4A-B. Pathway analysis derived by comparison of the relative abundance of metabolites from sera derived from patients with locoregional CRC and liver-only metastases, as determined by GC-MS. More centrally located molecules in the illustrated networks have a greater probability of participating in the biological processes involved in metastasis, but also represent hubs of diverse known biological functions. (FIG. 4A) The first network highlights the contribution of mediators of proliferation, apoptosis and energy consumption, as well as a prominent role of inflammatory mediators. As indicated, some of the molecules putatively involved are known for their contribution to the pathogenesis of metastasis in colorectal cancer. (FIG. 4B) The second network demonstrates that inflammatory processes are highly involved in the metastatic process.

### DETAILED DESCRIPTION

Biomarkers may be defined as any biomolecule or panel of biomolecules that can aid in the diagnosis of disease; prognostication; prediction of biology; or prediction of sensitivity to specific therapies. An objective of the work discussed herein was to determine whether, in patients with CRC, the serum metabolomic profile could be used as a biomarker to discriminate locoregional CRC from metastatic CRC; and to identify patients with liver-only metastases. The inventors used ¹H NMR spectroscopy because it is a well-established, robust and highly reproducible tool for obtaining a quantitative metabolomic profile of higher abundance metabolites. GC-MS was used to provide a more comprehensive metabolomic profile, and because it is a highly sensitive, rapid and accurate instrument for the detection of lower abundance metabolites. Using a combination of ¹H NMR spectroscopy and gas chromatography-mass spectroscopy (GC-MS) to obtain a relatively comprehensive metabolomic characterization, they were able to determine that patients with locoregional CRC, liver-only metastases, and extrahepatic metastases could, in fact, be discriminated using each of these approaches. These and other aspects of the invention are discussed in detail below.

### I. COLORECTAL CANCER

Colorectal cancer, commonly known as bowel cancer, is a cancer from uncontrolled cell growth in the colon (large intestine, rectum, or appendix). Symptoms typically include rectal bleeding and anemia which are sometimes associated with weight loss and changes in bowel habits. Most colorectal cancer occurs due to lifestyle and increasing age with only a minority of cases associated with underlying genetic disorders. It typically starts in the lining of the bowel and if left untreated, can grow into the deeper muscle layers, and then through the bowel wall. Screening is effective at decreasing the chance of dying from colorectal cancer and is recommended starting at the age of 50 and continuing until a person is 75-years old. Localized bowel cancer is usually diagnosed through sigmoidoscopy or colonoscopy.

Cancers that are confined within the wall of the colon are often curable with surgery while cancer that has spread widely around the body is usually not curable and management then focuses on extending the person's life via chemotherapy and improving quality of life. Colorectal cancer is the fourth most commonly diagnosed cancer in the world, but it is more common in developed countries. Around 60% of cases were diagnosed in the developed world. It is estimated that worldwide, in 2008, 1.23 million new cases of colorectal cancer were clinically diagnosed, and that it killed 608,000 people.

The symptoms and signs of colorectal cancer depend on the location of tumor in the bowel, and whether it has spread elsewhere in the body (metastasis). The classic warning signs include: worsening constipation, blood in the stool, weight loss, fever, loss of appetite, and nausea or vomiting in someone over 50 years old. While rectal bleeding or anemia are high-risk features in those over the age of 50, other commonly described symptoms including weight loss and change in bowel habit are typically only concerning if associated with bleeding.

Greater than 75-95% of colon cancer occurs in people with little or no genetic risk. While some risk factors such as older age and male gender cannot be changed many can. High fat, alcohol or red meat intake are risk factors for colorectal cancer as is obesity, smoking and a lack of physical exercise. The risk for alcohol appears to increase at greater than one drink per day. People with inflammatory bowel disease (ulcerative colitis and Crohn's disease) are at increased risk of colon cancer. The risk is greater the longer a person has had the disease, and the worse the severity of inflammation. In these high risk groups both prevention with aspirin and regular colonoscopies are recommended. People with inflammatory bowel disease account for less than 2% of colon cancer cases identified each year. In those with Crohn's disease, 2% get colorectal cancer after 10 years, 8% after 20 years, and 18% after 30 years. In those with ulcerative colitis approximately 20% develop tumors within the first 10 years.

Those with a family history in two or more first degree relatives have a two- to three-fold greater risk of disease and this group accounts for about 20% of all cases. A number of genetic syndromes are also associated with higher rates of colorectal cancer. The most common of these is hereditary nonpolyposis colorectal cancer (HNPCC or Lynch syndrome) which is present in about 3% of people with colorectal cancer. Other syndromes that are strongly associated include: Gardner syndrome, and familial adenomatous polyposis (FAP) in which cancer nearly always occurs and is the cause of 1% of cases.

Colorectal cancer is a disease originating from the epithelial cells lining the colon or rectum of the gastrointestinal tract, most frequently as a result of mutations in the Wnt signaling pathway that artificially increase signaling activity. The mutations can be inherited or are acquired, and must probably occur in the intestinal crypt stem cell. The most commonly mutated gene in all colorectal cancer is the APC gene, which produces the APC protein. The APC protein is a “brake” on the accumulation of β-catenin protein; without APC, β-catenin accumulates to high levels and translocates (moves) into the nucleus, binds to DNA, and activates the transcription of genes that are normally important for stem cell renewal and differentiation but when inappropriately expressed at high levels can cause cancer. While APC is mutated in most colon cancers, some cancers have increased β-catenin because of mutations in β-catenin (CTNNB1) that block its degradation, or they have mutation(s) in other genes with function analogous to APC such as AXIN1, AXIN2, TCF7L2, or NKD1.

Beyond the defects in the Wnt-APC-beta-catenin signaling pathway, other mutations must occur for the cell to become cancerous. The p53 protein, produced by the TP53 gene, normally monitors cell division and kills cells if they have Wnt pathway defects. Eventually, a cell line acquires a mutation in the TP53 gene and transforms the tissue from an adenoma into an invasive carcinoma. Other apoptotic proteins commonly deactivated in colorectal cancers are TGF-β and DCC (Deleted in Colorectal Cancer). TGF-β has a deactivating mutation in at least half of colorectal cancers. Sometimes TGF-β is not deactivated, but a downstream protein named SMAD is. DCC commonly has deletion of its chromosome segment in colorectal cancer.

Genes encoding the proteins KRAS, RAF and PI3K, which normally stimulate the cell to divide in response to growth factors, can acquire mutations that result in over-activation of cell proliferation. The chronological order of mutations is sometimes important, with a primary KRAS mutation generally leading to a self-limiting hyperplastic or borderline lesion, but if occurring after a previous APC mutation it often progresses to cancer. PTEN, a tumor suppressor, normally inhibits PI3K, but can sometimes become mutated and deactivated.

Diagnosis of colorectal cancer is via tumor biopsy typically done during sigmoidoscopy or colonoscopy. The extent of the disease is then usually determined by a CT scan of the chest, abdomen and pelvis. There are other potential imaging test such as PET and MRI which may be used in certain cases. Colon cancer staging is done next and based on the TNM system which is determined by how much the initial tumor has spread, if and where lymph nodes are involved and, and if and how many metastasis there are.

More than 80% colorectal cancers arise from adenomatous polyps making this cancer amenable to screening. The three main screening tests are fecal occult blood testing, flexible sigmoidoscopy and colonoscopy. Fecal occult blood testing of the stool is typically recommended every two years and can be either guaiac based or immunochemical. In the United States screening is recommended between the age of 50 and 75 years with sigmoidoscopy every 5 years and colonoscopy every 10 years. For those at high risk, screenings usually begin at around 40. Virtual colonoscopy via a CT scan appears almost as good as standard colonoscopy but is expensive and associated with radiation exposure. It is believed that screening has the potential to reduce colorectal cancer deaths by 60%. For people over 75 or those with a life expectancy of less than 10 years screening is not recommended.

The treatment of colorectal cancer depends on how advanced it is. When colorectal cancer is caught early surgery can be curative. However, when it is detected at later stages (metastases are present), this is less likely and treatment is often directed more at extending life and keeping people comfortable.

For people with localized cancer the preferred treatment is complete surgical removal with the attempt of achieving a cure. This can either be done by an open laparotomy or sometimes laparoscopically. If there are only a few metastases in the liver or lungs they may also be removed. Sometimes chemotherapy is used before surgery to shrink the cancer before attempting to remove it. The two most common sites of recurrence if it occurs is in the liver and lungs.

For locoregional disease, chemotherapy may be used in addition to surgery in certain cases as adjuvant therapy. If cancer has entered the lymph nodes adding chemotherapy agents such as 5-fluorouracil, capecitabine±oxaliplatin increases life expectancy. If the lymph nodes do not contain cancer the benefits of chemotherapy are controversial. If the cancer is widely metastatic or unresectable, treatment is then palliative. Typically in this case a number of different chemotherapy medications are used. Chemotherapy drugs may include combinations of agents including 5-fluorouracil, capecitabine, UFT, leucovorin, irinotecan, or oxaliplatin. While a combination of radiation and chemotherapy may be useful for rectal cancer, its use in colon cancer is not routine due to the sensitivity of the bowels to radiation.

In people with incurable colorectal cancer, palliative care can be considered for improving quality of life. Surgical options may include non-curative surgical removal of some of the cancer tissue, bypassing part of the intestines, or stent placement. These procedures can be considered to improve symptoms and reduce complications such as bleeding from the tumor, abdominal pain and intestinal obstruction. Non-operative methods of symptomatic treatment include radiation therapy to decrease tumor size as well as pain medications.

In Europe the 5-year survival for colorectal cancer is less than 60%. In the developed world, about a third of people who get the disease die from it. Survival is directly related to stage of disease, but overall is poor for symptomatic cancers, as they are typically quite advanced. Survival rates for early stage detection is about 5 times that of late stage cancers. For example, patients with a tumor that has not breached the muscularis mucosa (TNM stage Tis, N0, M0) have an average 5-year survival of 100%, while those with an invasive cancer, i.e., T1 (within the submucosal layer) or T2 (within the muscular layer) cancer have an average 5-year survival of approximately 90%. Those with a more invasive tumor, yet without node involvement (T3-4, N0, M0) have an average 5-year survival of approximately 70%. Patients with positive regional lymph nodes (any T, N1-3, M0) have an average 5-year survival of approximately 40%, while those with distant metastases (any T, any N, M1) have an average 5-year survival of approximately 5%.

According to the American Cancer Society statistics in 2006, over 20% of patients present with metastatic (stage IV) colorectal cancer at the time of diagnosis, and up to 25% of this group will have isolated liver metastasis that is potentially resectable. Lesions which undergo curative resection have demonstrated 5-year survival outcomes now exceeding 50%.

The aims of follow-up are to diagnose, in the earliest possible stage, any metastasis or tumors that develop later, but did not originate from the original cancer (metachronous lesions). The U.S. National Comprehensive Cancer Network and American Society of Clinical Oncology provide guidelines for the follow-up of colon cancer. A medical history and physical examination are recommended every 3 to 6 months for 2 years, then every 6 months for 5 years. Carcinoembryonic antigen blood level measurements follow the same timing, but are only advised for patients with T2 or greater lesions who are candidates for intervention. A CT-scan of the chest, abdomen and pelvis can be considered annually for the first 3 years for patients who are at high risk of recurrence (for example, patients who had poorly differentiated tumors or venous or lymphatic invasion) and are candidates for curative surgery (with the aim to cure). A colonoscopy can be done after 1 year, except if it could not be done during the initial staging because of an obstructing mass, in which case it should be performed after 3 to 6 months. If a villous polyp, a polyp >1 centimeter or high grade dysplasia is found, it can be repeated after 3 years, then every 5 years. For other abnormalities, the colonoscopy can be repeated after 1 year. Routine PET or ultrasound scanning, chest X-rays, complete blood count or liver function tests are not recommended. These guidelines are based on recent meta-analyses showing intensive surveillance and close follow-up can reduce the 5-year mortality rate from 37% to 30%.

### II. MEASUREMENT OF METABOLIC BIOMARKERS

### A. Subjects, Metabolites and Samples

As described herein, this inventor relates to methods and materials for assessing metabolites in a mammal that has or is suspected of having colorectal cancer. The mammal can be any type of mammal including, without any limitation, a mouse, rat, dog, cat, horse, sheep, goat, cow, pig, monkey, or human. A biological sample, from which the analysis is obtained, can be any biological specimen that contains relevant amounts of the biomarkers. Typically, the sample will be a fluid sample, such as blood, serum, plasma, or urine.

This document relates to the use of metabolite biomarkers for assessing metastatic disease presence and location in a mammal that has or is suspected of having colorectal cancer. As described herein, the levels of one or more of the biomarkers selected from those set forth in Tables 2A-D, below. Any combination of the biomarkers listed above can be used. For example, the methods can determine the level of one or more, e.g., two or more, three or more, four or more, five or more, six or more, seven or more, eight, nine, ten, fifteen, twenty, twenty-five, thirty or all of the biomarkers in Tables 2A-D; and comparing the levels of the biomarkers with reference levels of the same biomarkers. The level(s) of the biomarkers are compared to a reference, wherein the biomarkers' level in comparison to the reference is indicative of metastatic status (e.g., liver-only metastasis v. locoregional or extrahepatic metastasis).

A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. A “reference level” may also be a “standard curve reference level” based on the levels of one or more biomarkers determined from a population and plotted on appropriate axes to produce a reference curve (e.g., a standard probability curve). Reference levels may also be tailored to specific techniques that are used to measure levels of biomarkers in biological samples (e.g., LC-MS, GC-MS, NMR, enzyme assays, etc.), where the levels of biomarkers may differ based on the specific technique that is used.

In some cases, the reference comprises predetermined values for a plurality of biomarkers (e.g., each of the plurality of biomarkers). The predetermined value can take a variety of forms. It can be a level of a biomarker in a control mammal (e.g., a mammal with a particular type of metastatic disease). A predetermined value that represents a level(s) of a biomarker referred to herein as a predetermined level. A predetermined level can be single cut-off value, such as a median or mean. It can be a range of cut-off (or threshold) values, such as a confidence interval.

Mammals associated with predetermined values are typically referred to as control mammals (or controls). A control mammal may or may not have metastatic disease. Thus, in some cases the level of a biomarker in a mammal being greater than or equal to the level of the biomarker in a control mammal is indicative of a clinical status. In other cases the level of a biomarker in a mammal being less than or equal to the level of the biomarker in a control mammal is indicative of clinical status. The amount of the greater than and the amount of the less than is usually of a sufficient magnitude to, for example, facilitate distinguishing a mammal from a control mammal using the disclosed methods. Typically, the greater than, or the less than, that is sufficient to distinguish a mammal from a control mammal is a statistically significant greater than, or a statistically significant less than. In cases where the level of a biomarker in a mammal being equal to the level of the biomarker in a control mammal is indicative of a clinical status, the “being equal” refers to being approximately equal (e.g., not statistically different).

The predetermined value can depend upon a particular population of mammals selected. For example, an apparently healthy population will have a different ‘normal’ range of biomarkers than will a population of mammals which have, or are likely to have, metastatic disease. Accordingly, the predetermined values selected may take into account the category (e.g., healthy, at risk, diseased) in which a mammal falls. Appropriate ranges and categories can be selected with no more than routine experimentation by those of ordinary skill in the art.

In some cases a predetermined value of a biomarker is a value that is the average for a population of healthy mammals (e.g., human subjects who have no apparent signs and symptoms of an insulin sensitivity disorder). The predetermined value will depend, of course, on the particular biomarker selected and even upon the characteristics of the population in which the mammal lies.

### B. Analytical Methods

The levels of the biomarkers from a biological sample from a mammal can be obtained by any art recognized method. Typically, the level is determined by measuring the level of the biomarker in a body fluid (clinical sample), e.g. blood, plasma, or urine. The level can be determined by any method known in the art, e.g. immunoassays, enzymatic assays, mass-spectrometry (MS), tandem-mass-spectrometry (MS-MS), Liquid chromatography-mass spectrometry (LC-MS), high performance liquid chromatography (HPLC), Ultra-Performance Liquid Chromatography (UPLC), nuclear magnetic resonance (NMR) spectroscopy, infrared (IR) spectroscopy, gas chromatography (GC) or other known techniques for determining the presence and/or quantity of a metabolite. Conventional methods include sending clinical sample(s) to a commercial laboratory for measurement or the use of commercially available assay kits.

Gas chromatography-mass spectrometry (GC-MS) is a method that combines the features of gas-liquid chromatography and mass spectrometry to identify different substances within a test sample. Applications of GC-MS include drug detection, fire investigation, environmental analysis, explosives investigation, and identification of unknown samples. GC-MS can also be used in airport security to detect substances in luggage or on human beings. Additionally, it can identify trace elements in materials that were previously thought to have disintegrated beyond identification.

1. GC-MS GC-MS has been widely heralded as a “gold standard” for forensic substance identification because it is used to perform a specific test. A specific test positively identifies the actual presence of a particular substance in a given sample. A non-specific test merely indicates that a substance falls into a category of substances. Although a non-specific test could statistically suggest the identity of the substance, this could lead to false positive identification.

The GC-MS is composed of two major building blocks: the gas chromatograph and the mass spectrometer. The gas chromatograph utilizes a capillary column which depends on the column's dimensions (length, diameter, film thickness) as well as the phase properties (e.g., 5% phenyl polysiloxane). The difference in the chemical properties between different molecules in a mixture will separate the molecules as the sample travels the length of the column. The molecules are retained by the column and then elute (come off of) from the column at different times (called the retention time), and this allows the mass spectrometer downstream to capture, ionize, accelerate, deflect, and detect the ionized molecules separately. The mass spectrometer does this by breaking each molecule into ionized fragments and detecting these fragments using their mass to charge ratio.

These two components, used together, allow a much finer degree of substance identification than either unit used separately. It is not possible to make an accurate identification of a particular molecule by gas chromatography or mass spectrometry alone. The mass spectrometry process normally requires a very pure sample while gas chromatography using a traditional detector (e.g., Flame ionization detector) cannot differentiate between multiple molecules that happen to take the same amount of time to travel through the column (i.e., have the same retention time), which results in two or more molecules that co-elute. Sometimes two different molecules can also have a similar pattern of ionized fragments in a mass spectrometer (mass spectrum). Combining the two processes reduces the possibility of error, as it is extremely unlikely that two different molecules will behave in the same way in both a gas chromatograph and a mass spectrometer. Therefore, when an identifying mass spectrum appears at a characteristic retention time in a GC-MS analysis, it typically lends to increased certainty that the analyte of interest is in the sample.

A mass spectrometer is typically utilized in one of two ways: full scan or selected ion monitoring (SIM). The typical GC-MS instrument is capable of performing both functions either individually or concomitantly, depending on the setup of the particular instrument.

The primary goal of instrument analysis is to quantify an amount of substance. This is done by comparing the relative concentrations among the atomic masses in the generated spectrum. Two kinds of analysis are possible, comparative and original. Comparative analysis essentially compares the given spectrum to a spectrum library to see if its characteristics are present for some sample in the library. This is best performed by a computer because there is a myriad of visual distortions that can take place due to variations in scale. Computers can also simultaneously correlate more data (such as the retention times identified by GC), to more accurately relate certain data.

Another method of analysis measures the peaks in relation to one another. In this method, the tallest peak is assigned 100% of the value, and the other peaks being assigned proportionate values. All values above 3% are assigned. The total mass of the unknown compound is normally indicated by the parent peak. The value of this parent peak can be used to fit with a chemical formula containing the various elements which are believed to be in the compound. The isotope pattern in the spectrum, which is unique for elements that have many isotopes, can also be used to identify the various elements present. Once a chemical formula has been matched to the spectrum, the molecular structure and bonding can be identified, and must be consistent with the characteristics recorded by GC-MS. Typically, this identification done automatically by programs which come with the instrument, given a list of the elements which could be present in the sample.

A “full spectrum” analysis considers all the “peaks” within a spectrum. Conversely, selective ion monitoring (SIM) only monitors selected peaks associated with a specific substance. This is done on the assumption that at a given retention time, a set of ions is characteristic of a certain compound. This is a fast and efficient analysis, especially if the analyst has previous information about a sample or is only looking for a few specific substances. When the amount of information collected about the ions in a given gas chromatographic peak decreases, the sensitivity of the analysis increases. So, SIM analysis allows for a smaller quantity of a compound to be detected and measured, but the degree of certainty about the identity of that compound is reduced.

2. ¹H NMR Spectroscopy

Proton NMR (also Hydrogen-1 NMR, or ¹H NMR) is the application of nuclear magnetic resonance in NMR spectroscopy with respect to hydrogen-1 nuclei within the molecules of a substance, in order to determine the structure of its molecules. In samples where natural hydrogen (H) is used, practically all of the hydrogen consists of the isotope ¹H (hydrogen-1; i.e., having a proton for a nucleus). A full ¹H atom is called protium.

Simple NMR spectra are recorded in solution, and solvent protons must not be allowed to interfere. Deuterated (deuterium=²H, often symbolized as D) solvents especially for use in NMR are preferred, e.g., deuterated water, D2O, deuterated acetone, (CD₃)₂CO, deuterated methanol, CD₃OD, deuterated dimethyl sulfoxide, (CD₃)2SO, and deuterated chloroform, CDCl₃. However, a solvent without hydrogen, such as carbon tetrachloride, CCl₄ or carbon disulphide, CS₂, may also be used.

Historically, deuterated solvents were supplied with a small amount (typically 0.1%) of tetramethylsilane (TMS) as an internal standard for calibrating the chemical shifts of each analyte proton. TMS is a tetrahedral molecule, with all protons being chemically equivalent, giving one single signal, used to define a chemical shift=0 ppm. It is volatile, making sample recovery easy as well. Modern spectrometers are able to reference spectra based on the residual proton in the solvent (e.g., the CHCl3, 0.01% in 99.99% CDCl₃). Deuterated solvents are now commonly supplied without TMS.

Deuterated solvents permit the use of deuterium frequency-field lock (also known as deuterium lock or field lock) to offset the effect of the natural drift of the NMR's magnetic field. In order to provide deuterium lock, the NMR constantly monitors the deuterium signal resonance frequency from the solvent and makes changes to the to keep the resonance frequency constant. Additionally, the deuterium signal may be used to accurately define 0 ppm as the resonant frequency of the lock solvent and the difference between the lock solvent and 0 ppm (TMS) are well known.

Proton NMR spectra of most organic compounds are characterized by chemical shifts in the range +14 to −4 ppm and by spin-spin coupling between protons. The integration curve for each proton reflects the abundance of the individual protons. Simple molecules have simple spectra. The spectrum of ethyl chloride consists of a triplet at 1.5 ppm and a quartet at 3.5 ppm in a 3:2 ratio. The spectrum of benzene consists of a single peak at 7.2 ppm due to the diamagnetic ring current.

Chemical shift values, symbolized by δ, are not precise, but typical—they are to be therefore regarded mainly as orientational. Deviations are in ±0.2 ppm range, sometimes more. The exact value of chemical shift depends on molecular structure and the solvent in which the spectrum is being recorded. Hydrogen nuclei are sensitive to the hybridization of the atom to which the hydrogen atom is attached and to electronic effects. Nuclei tend to be deshielded by groups which withdraw electron density. Deshielded nuclei resonate at higher δ values, whereas shielded nuclei resonate at lower δ values.

Examples of electron withdrawing substituents are —OH, —OCOR, —OR, —NO₂ and halogens. These cause a downfield shift of approximately 2-4 ppm for H atoms on Cα and of less than 1-2 ppm for H atoms on Cβ. Cα is an aliphatic C atom directly bonded to the substituent in question, and Cβ is an aliphatic C atom bonded to Cα. Carbonyl groups, olefinic fragments and aromatic rings contribute sp2 hybridized carbon atoms to an aliphatic chain. This causes a downfield shift of 1-2 ppm at Cα.

Note that labile protons (—OH, —NH₂, —SH) have no characteristic chemical shift. However such resonances can be identified by the disappearance of a peak when reacted with D₂O, as deuterium will replace a protium atom. This method is called a D₂O shake. Acidic protons may also be suppressed when a solvent containing acidic deuterium ions (e.g., methanol-d4) is used.

The chemical shift is not the only indicator used to assign a molecule. Because nuclei themselves possess a small magnetic field, they influence each other, changing the energy and hence frequency of nearby nuclei as they resonate—this is known as spin-spin coupling. The most important type in basic NMR is scalar coupling. This interaction between two nuclei occurs through chemical bonds, and can typically be seen up to three bonds away.

The effect of scalar coupling can be understood by examination of a proton which has a signal at 1 ppm. This proton is in a hypothetical molecule where three bonds away exists another proton (in a CH—CH group for instance), the neighbouring group (a magnetic field) causes the signal at 1 ppm to split into two, with one peak being a few hertz higher than 1 ppm and the other peak being the same number of hertz lower than 1 ppm. These peaks each have half the area of the former singlet peak. The magnitude of this splitting (difference in frequency between peaks) is known as the coupling constant. A typical coupling constant value would be 7 Hz.

The coupling constant is independent of magnetic field strength because it is caused by the magnetic field of another nucleus, not the spectrometer magnet. Therefore it is quoted in hertz (frequency) and not ppm (chemical shift).

In another molecule a proton resonates at 2.5 ppm and that proton would also be split into two by the proton at 1 ppm. Because the magnitude of interaction is the same the splitting would have the same coupling constant 7 Hz apart. The spectrum would have two signals, each being a doublet. Each doublet will have the same area because both doublets are produced by one proton each.

### C. Kits

The document also provides kits for evaluating biomarkers in a mammal. The kits of the invention can take on a variety of forms. Typically, the kits will include reagents suitable for determining levels of one or more of the biomarkers disclosed herein. For example, the kits may contain one or more control samples. For example, the control samples can be specific for levels of one or more biomarkers that correspond to various forms of metastatic disease. Typically, a comparison between the levels of the biomarkers in the sample from the mammal and the levels of the biomarkers in the control samples is indicative of a clinical status.

Also, the kits, in some cases, will include written information (indicia) providing a reference (e.g., predetermined values), wherein a comparison between the levels of the biomarkers in the subject and the reference (pre-determined values) is indicative of a clinical status. In some cases, the kits comprise software useful for comparing biomarker levels with a reference (e.g., a prediction model). Usually the software will be provided in a computer readable format such as a compact disc, but it also may be available for downloading via the internet. However, the kits are not so limited and other variations will be apparent to one of ordinary skill in the art.

### III. EXAMPLES

The following examples are included to demonstrate particular embodiments of the invention. It should be appreciated by those of skill in the art that the techniques disclosed in the examples which follow represent techniques discovered by the inventor to function well in the practice of the invention. However, those of skill in the art, should in light of the present disclosure, appreciate that many changes can be made in the specific embodiments which are disclosed and still obtain a like or similar result without departing from the spirit and scope of the invention.

### Example 1

### Methods

Sample Collection

Clinically annotated serum samples were collected from consented patients who underwent surgery for resection of their primary colorectal adenocarcinoma, resection of liver metastases, or resection of extrahepatic metastases. All patients were treated at the Foothills Medical Centre, a tertiary referral centre, between 2004 and 2009. Patients with any acute inflammation or sepsis were specifically excluded. Surgical pathology was reviewed for all patients, and confirmed all had colorectal adenocarcinoma. Samples were collected in a plastic gold top Vacutainer tube (BD Biosciences), which contained a clot activator and a gel for serum separation. Samples were processed within 6 hours of collection, then frozen at −20° C. until the time of analysis. All samples were collected from patients who had fasted, prior to surgery.

¹H NMR Spectrometry.

¹H NMR spectroscopy was performed as previously described (Bathe et al., 2011). Briefly, all experiments were performed on a Bruker Avance 600 NMR spectrometer (Bruker Biospin, Milton, Canada) operating at 600.22 MHz and equipped with a 5 mm TXI probe at 298 K. One-dimensional ¹H NMR spectra were obtained using a standard Bruker pulse sequence program (Bruker pr1 d_noesy). Spectra were acquired as series of 1024 scans, and then Fourier transformed using the Chenomx NMRSuite processor module in 65536 data-points over spectral width of 7211 Hz. A line broadening of 0.5 Hz was applied to all spectra before a standard procedure of phasing, B-spline baseline correction, water deletion, and reference deconvolution with DSS peak calibration using the Chenomx NMRSuite processor module. Metabolites were assigned based on comparison of both ¹H and ¹³C chemical shifts and spin-spin coupling constants with those of model compounds in Human Metabolome Database (HMDB, version 2.5) (Wishart et al., 2009) and Chenomx NMR Suite 6.1 software (Chenomx Inc., Edmonton, Canada). Metabolites were quantified using the targeted profiling approach Weljie et al., 2006) as implemented in the Chenomx software. Metabolite intensities were integrally normalized against the sum of the metabolites' intensities for each sample, to adjust for possible inter-sample concentration variations.

GC-MS Spectrometry.

The methods of Bligh and Dyer were used for metabolite extraction (Bligh and Dyer, 1959). Briefly, layers of the two-phase mixture of chloroform and methanol were transferred to individual tubes. Aqueous layer tubes were dried under vacuum (SpeedVac, Eppendorf, Germany) and stored at −20° C. until derivatization. For metabolite derivatization, 50 μL of methoxyamine-hydrochloride in pyridine solution (20 mg/ml) was added to each tube. N-Methyl-N-(trimethylsilyl)trifluoroacetamide (MSTFA; Sigma-Aldrich, Germany) was added as silylating agent. Samples were diluted with hexane, and tubes were centrifuged to remove any solid particles and micro-particles. Ultimately 200 μL of supernatant were transferred to a GC-MS vial with glass inserts, in preparation for GC injection.

GC-MS was performed on an Agilent chromatograph 7890A (Agilent Technologies, USA) coupled with a Waters GCT mass spectrometer, using GC-TOF-MS methodology. MS was operated in a range of 50-800 m/z. Mass spectra were processed using Metabolite Detector software (Version 2.06, Technische Universität Carolo-Wilhelmina zu Braunschweig, Braunschweig, Germany). For metabolite identification, the GOLM metabolite database (Hummel et al., 2007) and NIST 2008 library (Stein, 1995) were used. Identified peak intensities were integrally normalized against the sum of the peak intensities for each sample as the last step in the data pre-processing.

Data Analysis.

Patients were allocated to one of three groups, based on stage and site of disease. Descriptive statistics were utilized to characterize the groups, with unpaired t-tests with unequal variances assumed (Welch's t-test) used to compare means and Fisher's exact tests used to compare categorical variables. All tests of significance were 2-sided and a p value<0.05 was considered a priori to represent statistical significance between groups of patients in these univariate analyses.

Normalized data were log transformed, centered and unit variance scaled, then analyzed using the SIMCA-P+ program (Version 12.0, Umetrics AB, Umeå, Sweden). Pairwise comparisons between the three groups were carried out using the same modeling approach, separately for each metabolomic platform. First, a preliminary principal component analysis (PCA) model was carried out including up to three components per PCA. This was done primarily to identify the potential variables which could form distinct sample subsets and intrinsic patterns, and to detect potential outlier samples (data not shown). Next, selection of potentially important metabolites was carried out using two sample t-tests which assumed unequal variances. A p-value threshold of 0.3 was used to select these potentially important metabolites for inclusion in the supervised orthogonal partial least squares discriminate analyses (O-PLS-DA). In previous work, the inventors have demonstrated that using this filtering approach to reduce the number of metabolites to those that are potentially informative results in a high degree of concordance between p-values obtained from univariate comparisons and variable influence on projection (VIP) values in OPLS-DA models (Bathe et al., 2011; Weljie et al., 2007).

An alternative approach to t-test filtering to identify potentially important metabolites was carried out using stability selection (Wehrens et al., 2011). This strategy utilizes PLS-DA models to evaluate the stability of the metabolites under data perturbations, which is advantageous when the number of samples is not large. Analyses were conducted using the R package, BioMark (R Development Core Team, 2013), using the same normalized, log transformation, centered and unit variance scaled data. Metabolites selected at least 50% of the time in 100 perturbations of the data were identified, where each perturbation randomly omitted 10% of the samples from each group and randomly selected 70% of the metabolites. The lists of selected metabolites were subsequently evaluated using OPLS-DA models.

Three performance measures were used to assess the final OPLS-DA models: CV-ANOVA for assessing their reliability; R²Y, which describes the fraction of the explained variation by the non-orthogonal component for the group status variable; and Q²Y, which is a measure of the predictability of the model. This predicted fraction was determined through a seven-fold cross validation during the model-building process by leaving a seventh of samples out of every round and then testing the model against the remaining portion of data. R²Y and Q²Y scores range between 0 and 1, where R²Y score of 1 demonstrates that 100% of variance is explained by the model, and Q²Y score closer to 1 indicates higher reliability of the prediction in cross-validation procedure. R² score is always larger than Q² score, but an observed difference of more than 0.3 between the R² and Q² scores should be carefully examined. Potential confounders (age, gender, and chemotherapy within 3 months of sample collection) were examined for their importance in our multivariate regression models. Receiver-operating characteristic curves were used to provide summaries of the predictive performance of constructed models (Metz-ROC, University of Chicago, Ill., USA 2011).

Internal Verification of Clinical Applicability.

The Receiver Operating Characteristic (ROC) curve is an indicator of the predictive performance of a developed test and depicts the range of relationships between sensitivity and specificity. In this study, the inventors tested the predictive performance of our discriminant models to distinguish between pairs of disease states (locoregional disease, liver-only metastases, and extrahepatic metastases) by constructing seven models with one seventh of the data excluded from each model, and with each sample excluded once. The ability of the average of the seven models to predict the excluded samples provided a measure of the predictive ability of each metabolomic profiling model. Using these average predicted group values (Ypredcv from the Umetrics software), the inventors were able to generate a ROC for each comparison.

ROC curves were plotted for ¹H NMR spectroscopy and GC-MS to demonstrate the ability to predict the presence of liver-only metastases or locoregional CRC. Values greater than 0.8 indicate excellent predictive ability.

Pathway Analysis.

Known differentially abundant and co-regulated components in GC-MS analysis (on supervised OPLS-DA) were used for metabolite pathway analysis using Metaboanalyst Version 2.0 (Xia et al., 2009). This web-based software enables identification of altered metabolic pathways from its extensive HMDB-derived collection of more than 70 pathways and metabolite libraries.

Network and pathway analyses were generated using the Ingenuity Pathways Analysis program (Ingenuity® Systems, world-wide-web at ingenuity.com). A dataset containing chemical KEGG identifiers of the same components was uploaded into the program, one for each comparison. Each identifier renders a pertinent metabolite in Ingenuity's Knowledge Base, generating a network eligible metabolites list. These metabolites were then projected onto Ingenuity's Knowledge based global metabolite network. Subsequently, networks of these eligible molecules were algorithmically generated by IPA, based on their connectivity using functional core analysis.

### Example 2

### Results

Patients and Demographics.

Patients with pathologically confirmed CRC who were potential candidates for surgery were included in the analysis. Sera were collected under standard fasting conditions. Patients were assigned to three groups: locoregional CRC (stages II and III; Group 1; N=41); liver-only metastases (Group 2; N=44; and extrahepatic metastases (Group 3; N=26). All patients with locoregional CRC and with liver-only metastases underwent resection. Patients with extrahepatic metastases underwent various surgical procedures to remove or to debulk all grossly apparent disease.

The characteristics of each patient group are summarized in Table 1. There were a number of differences in the groups which were evaluated in the multivariate model analyses to determine the effects of these covariables. Patients in the groups with metastatic disease were significantly younger on average than patients with locoregional CRC (P=0.004), but there was no significant difference in average age between patients with liver-only metastases and extrahepatic metastases. There was a higher proportion of males in Group 2, compared to Group 1, but Groups 2 and 3 had similar gender distributions. Chemotherapy was more frequently given within 3 months of sample collection in patients with metastatic disease. However, there was no statistical difference in the proportion of patients who had chemotherapy in Groups 2 and 3. All administered chemotherapy agents are listed in Table 1.

To evaluate the effects of each of the potential confounders (age, gender, exposure to chemotherapy within 3 months) on metabolomic profiles, we developed O2-PLS-DA regression models that included the effects of these factors in these models. All regression models revealed that none of these factors had significant confounding effects on the metabolomic profiles and so were not included in the final O-PLS-DA models.

TABLE 1
Patient Characteristics of Each Group

| | Locoregional | | Liver-only | | Extra-hepatic | | | | P |
|---|---|---|---|---|---|---|---|---|---|
| | CRC | | Metastases | | Metastases | | P | P | Locoregional |
| | Group 1 | | Group 2 | | Group 3 | | Group 1 vs | Group 2 | CRC vs. All |
| | (N = 42) | | (N = 44) | | (N = 26) | | Group 2 | vs Group 3 | Stage IV |
| Age, y | 72 ± 11 | | 67 ± 13 | | 63 ± 13 | | 0.05 | 0.19 | 0.004 |
| Gender | | | | | | | 0.06 | 0.50 | 0.39 |
| Male | 21 | (50) | 31 | (69) | 15 | (60) | | | |
| Female | 21 | (50) | 14 | (31) | 10 | (40) | | | |
| Bowel Prep | 39 | (100) | 45 | (100) | 25 | (100) | NS | NS | NS |
| Stage | | | | | | | | | |
| Stage II | 21 | | 0 | | 0 | | | | |
| Stage III | 21 | | 0 | | 0 | | | | |
| Stage IV | 0 | | 45 | | 25 | | | | |
| Any Chemotherapy within 3 mo. | 5 | (21) | 16 | (36) | 9 | (36) | 0.02 | 0.79 | 0.02 |
| Specific Chemotherapeutic | | | | | | | | | |
| Agents | | | | | | | | | |
| 5-FU | 5 | (100) | 15 | (94) | 7 | (78) | | | |
| Oxaliplatin | 1 | (20) | 7 | (44) | 4 | (44) | | | |
| Irinotecan | 0 | (0) | 10 | (63) | 2 | (22) | | | |
| Bevacizumab | 0 | (0) | 2 | (13) | 0 | (0) | | | |
| Other chemotherapy | 1 | (20) | 1 | (6) | 2 | (22) | | | |

Numbers in brackets represent percent (%).

Distinguishing Locoregional CRC from Liver-Only Metastases.

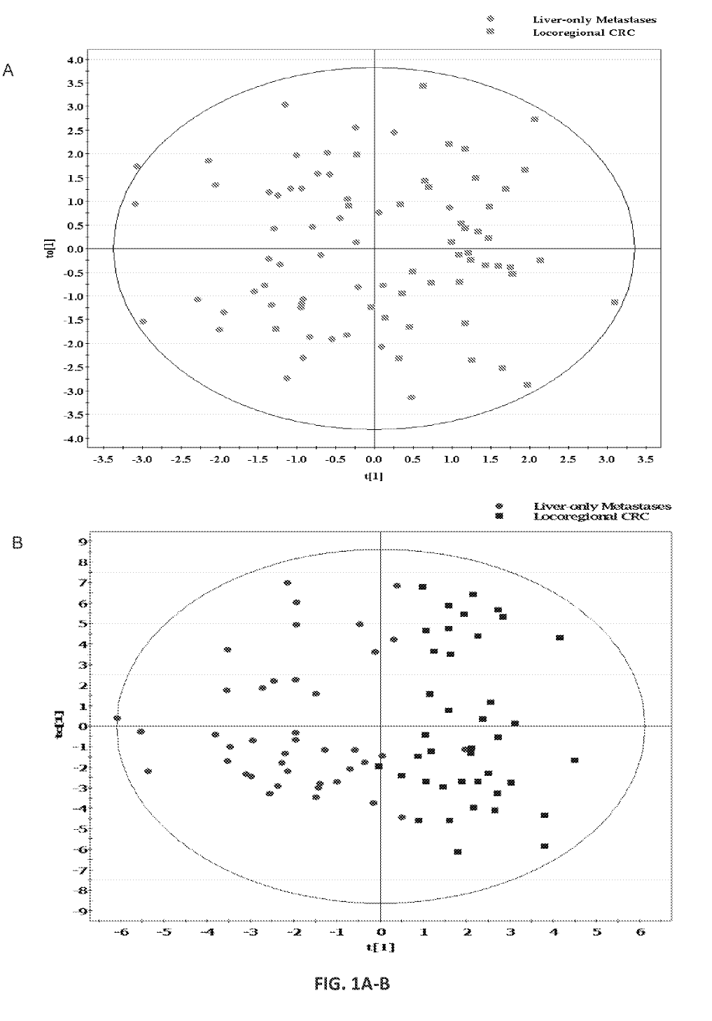

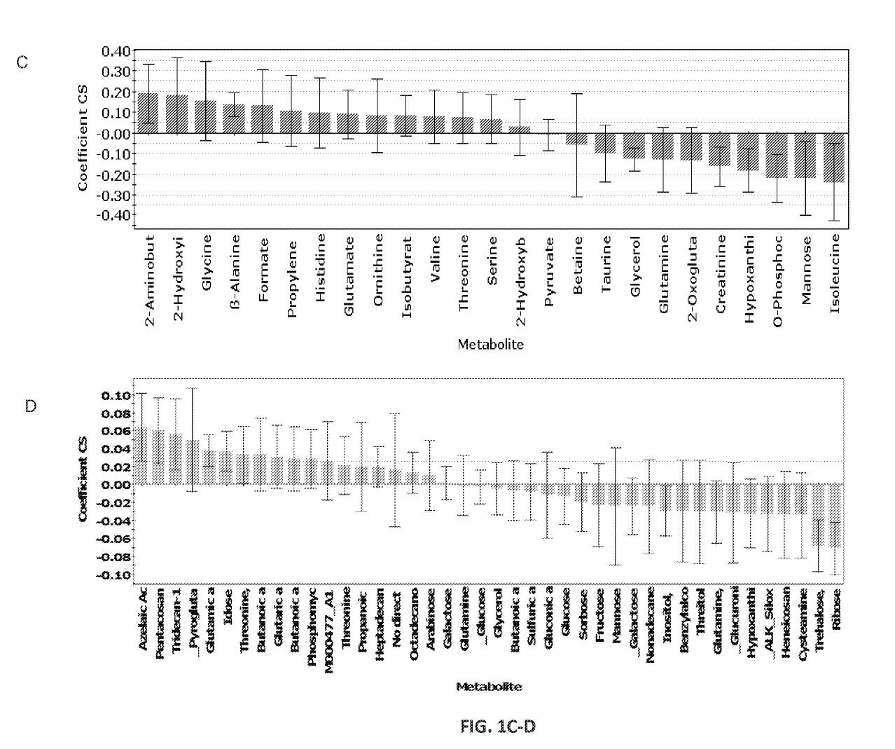

By ¹H NMR spectroscopy, 55 metabolites were detected with 25 found to be differentially abundant in the initial data filtering process, using a p-value less than 0.30. This cut-off was used to select only the potentially informative metabolites, to be included in subsequent supervised multivariate analysis (O-PLS-DA). By ¹H NMR spectroscopy alone, there was a very robust distinction between liver-only metastases and locoregional CRC(R²Y Score=0.61). The predictive ability of the model was measured by 7-fold cross validation (Q² score=0.39, CV-ANOVA p-value=5.10×10⁻⁷) (FIG. 1A). The coefficient plot demonstrating degree of differential abundance for each metabolite is depicted in FIG. 1C. To further refine the biomarker panel, we used the “stability selection” method (explained earlier). Briefly, the metabolites and samples are being left out in groups (jack-knifing) and the perturbation is analysed using the PLS-DA method. The metabolites are being ranked by the percentage of the iterations they have been in the “top fraction” of the features list by rank, in each iteration. The selected potential biomarkers are then being tested using OPLS-DA as our definitive method. Our final list consists of 15 core metabolites and 3 optional metabolites which could reach to an acceptable coverage of variance as well as prediction score (R²Y Score=0.59, Q²Y score=0.43, CV-ANOVA p-value=3.14×10⁻⁹, Area under the ROC curve=0.91). Our findings showed any model consisting the core set of 15 metabolites and one or more of the optional set will have Q2Y score of greater than 0.40 and AUROC of greater than 0.90.

GC-MS could detect 476 components across the entire range of samples, of which 170 were identified as metabolites. In total, following the initial data filtering process, 39 known metabolites and 114 unidentified components were found to be differentially abundant between patients with locoregional CRC and patients with liver-only metastases, using two sample t-tests with p-value cutoffs of 0.3. Orthogonal partial least squares discriminant analysis of 124 selected compounds found to be differentially abundant on the GC-MS platform also demonstrated that patients with liver-only metastases could be distinguished from patients with locoregional disease (R² score=0.68, Q² score=0.40, CV-ANOVA p-value=1.79×10⁻⁷) (FIG. 1B). To get the highest reproducibility and accuracy possible with the minimum number of mass spectrometry features, the list was further filtered based on each component's importance on the projection (VIP). Out of the initial 153 components, 26 were selected to form the core list and 16 additional ones to form the optional set. Models including the core set and one or more of the components in the optional list will have the Q²Y score of greater than 0.42 and the Area Under ROC curve exceeding the reliable threshold of 0.90 (CV-ANOVA p-value=9.42×10⁻⁸). Tables 2A-D provides a list of identified metabolites found by each analytical modality to be differentially abundant between patients with locoregional CRC and liver-only metastases.

TABLE 2A-D
Metabolites Found to be Differentially Abundant
in ¹H NMR and GC-MS in Pair of Patient Groups

| | Increased in liver- | Decreased in liver- |
|---|---|---|
| Group comparison, | limited metastases | limited metastases |
| analytical platform | Metabolite | Metabolite |
| TABLE 2A: | Core set | |
| Liver-only | 2-Aminobutyrate | Glycerol |
| disease versus | 2-Hydroxyisovalerate | 2-Oxoglutarate |
| locoregional | Formate | Glutamine |
| disease, 1H-NMR | Glutamate | Creatinine |
| | Isobutyrate | Hypoxanthine |
| | β-Alanine | Isoleucine |
| | Histidine | Mannose |
| | | O-Phosphocholine |
| | Optional set | |
| | Glycine | Acetate |
| | Pyruvate | |
| TABLE 2B: | Core set | |
| Liver-only | Unmatched: RI 2614.90 | Glucose (5TSM) BP |
| disease versus | Unmatched: RI 1581.92 | Glycerol (3TMS) |
| locoregional | Unmatched: RI 2405.88 | Glycerol (3TMS) |
| disease, GC-MS | Unmatched: RI 1708.86 | Galactose (5TMS) MP |
| | Unmatched: RI 1955.74 | Alkane RI 2465.91 |
| | Unmatched: RI 1722.29 | Unmatched: RI 1837.33 |
| | Unmatched: RI 1536.03 | Inositol, myo- |
| | Unmatched: RI 1570.18 | Glutamine, DL- |
| | Unmatched: RI 1103.53 | Unmatched: RI 2536.32 |
| | Alkane RI 1685.9 | Alkane RI 2582.98 |
| | Idose (5TMS) | Trehalose, alpha, |
| | | alpha-, D- |
| | | Ribose |
| | | Unmatched: RI 1692.50 |
| | | Unmatched: RI 1597.76 |
| | | Unmatched: RI 1652.02 |
| | Optional set | |
| | Azelaic Acid (2TMS) | Unmatched: RI 1103.97 |
| | Unmatched: RI 1890.31 | Unmatched: RI 1114.70 |
| | Unmatched: RI 2474.93 | Alkane RI 1416.54 |
| | Unmatched: RI 1603.68 | Unmatched: RI 1266.68 |
| | Unmatched: RI 1823.19 | Glucose (5TMS) MP |
| | Unmatched: RI 3182.13 | Glutamine, DL- (3TMS) |
| | Unmatched: RI 1166.61 | |
| | Serine (2TMS) | |
| | Unmatched: RI 1106.14 | |
| | Phosphoric acid (3TMS) | |
| TABLE 2C: | Core set | |
| Extrahepatic | Methionine | Glutamine |
| metastases versus | Tyrosine | Leucine |
| liver-only | Serine | Taurine |
| disease, 1H-NMR | Fumarate | Mannose |
| | Formate | 2-Oxoglutarate |
| | Glutamate | Isoleucine |
| | Optional set | |
| | Pyruvate | Arginine |
| TABLE 2D: | Core set | |
| Extrahepatic | Unmatched: RI 1693.97 | |
| metastases versus | Unmatched: RI 1890.31 | Glycerol (3TMS) |
| liver-only | Tridecan-1-ol, n- | Glucose |
| disease, GC-MS | Sulfuric acid | Alkane RI 1542.48 |
| | Pentadecan-1-ol, n- | Unmatched: RI 1114.70 |
| | Unmatched: RI 2090.22 | Alkane RI 1416.54 |
| | Unmatched: RI 1130.58 | Alkane RI 1020.15 |
| | Unmatched: RI 1416.93 | Unmatched: RI 1598.28 |
| | Unmatched: RI 2245.62 | Unmatched: RI 1035.78 |
| | Phenylalanine (2TMS) | |
| | Unmatched: RI 2408.20 | |
| | Alkane RI 2502.8 | |
| | Unmatched: RI 2024.61 | |
| | Unmatched: RI 2219.97 | |
| | Octadecadienoic acid, n- | |
| | Unmatched: RI 2823.31 | |
| | Unmatched: RI 1486.53 | |
| | Optional set | |
| | Alkane RI 1239.08 | Glucuronic acid |
| | Unmatched: RI 3156.28 | Unmatched: RI 1103.97 |
| | Unmatched: RI 1630.31 | Unmatched: RI 1007.71 |
| | Serine (2TMS), RI | Galactose |
| | 1253.99 | |
| | Alkane RI 2774.14 | Inositol, myo- |
| | | Benzene, 1,3-dichloro-, |
| | | RI 1022.1 |

Metabolites in the above tables are selected based on the two-sample t-test statistics used for data filtering (p-value smaller than 0.30) and/or the percentage of time selected using stability-based approach (fraction of time selected greater than 0.5) and the threshold of Variable Importance in the Projection (VIP) greater than 0.8. Correlation with the model comparison is determined by Scaled and Centered coefficients.

TABLE 3
Signatures of four models of Tables 2A-D and their performance

| Comparison | | No. of | R²Y | Q²Y | |
|---|---|---|---|---|---|
| and platform | Set | metabolites | score | score | AUROC |
| Liver-only | Core | 15 | 0.53 | 0.38 | 0.89 |
| disease versus | Core + | 18 | 0.59 | 0.43 | 0.91 |
| locoregional | Optional | | | | |
| disease, ¹H- | | | | | |
| NMR | | | | | |
| (TABLE 2A) | | | | | |
| Liver-only | Core | 26 | 0.57 | 0.42 | 0.90 |
| disease versus | Core + | 42 | 0.60 | 0.48 | 0.92 |
| locoregional | Optional | | | | |
| disease, GC- | | | | | |
| MS (TABLE | | | | | |
| 2B) | | | | | |
| Extrahepatic | Core | 12 | 0.36 | 0.17 | 0.77 |
| Metastases | Core + | 14 | 0.38 | 0.20 | 0.79 |
| versus liver- | Optional | | | | |
| only disease, | | | | | |
| ¹H-NMR | | | | | |
| (TABLE 2C) | | | | | |
| Extrahepatic | Core | 25 | 0.58 | 0.45 | 0.92 |
| Metastases | Core + | 36 | 0.65 | 0.57 | 0.94 |
| versus liver- | Optional | | | | |
| only disease, | | | | | |
| GC-MS | | | | | |
| (TABLE 2D) | | | | | |

The R2Y and Q2Y scores are calculated based on OPLS-DA method, using SIMCA 12 package (Bylesjö et al., 2006). The Area Under ROC is calculated by JLABROC4 by Charles Metz & colleagues in University of Chicago (Roe and Metz, 1997).

The inventors further analyzed the group with liver-only disease to derive information on the sensitivity of metabolomics-based tests for detection of liver metastases. Solitary metastases were present in 23 patients. These ranged in size from 14-99 mm in maximal diameter. Regression models revealed that number of liver lesions (solitary vs. multiple) did not have significant confounding effects on the metabolomic profiles. Indeed, when only patients with solitary nodules were included, metabolomic profiles remained different in the two stage groupings, by ¹H NMR spectroscopy (P=2.60×10⁻⁵) and by GC-MS (P=4.17×10⁻⁵).

To ensure that chemotherapy had no inadvertent effect on the inventors' ability to distinguish between locoregional disease and liver metastases, they excluded patients who had chemotherapy within 3 months of sample collection, and utilized the same models to compare these two groups. This confirmed that the metabolomic profiles were different in the two stage groupings, by ¹H NMR spectroscopy (P=5.32×10⁻⁶) and by GC-MS (P=0.006).

Distinguishing Liver-Only Metastasis from Extrahepatic Metastasis.

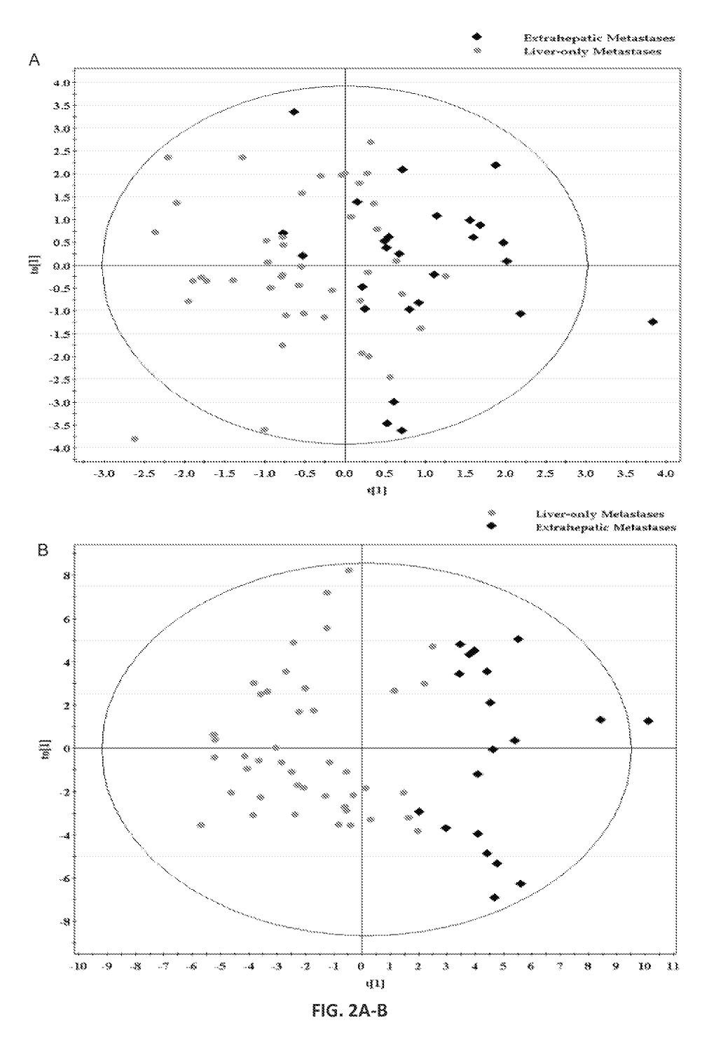

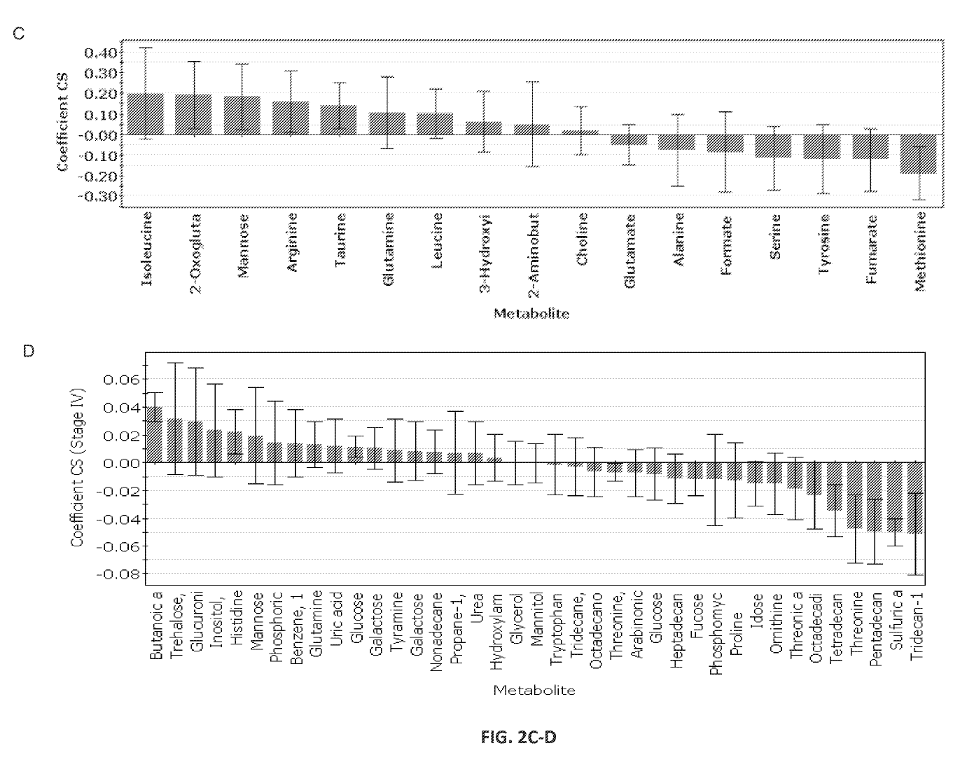

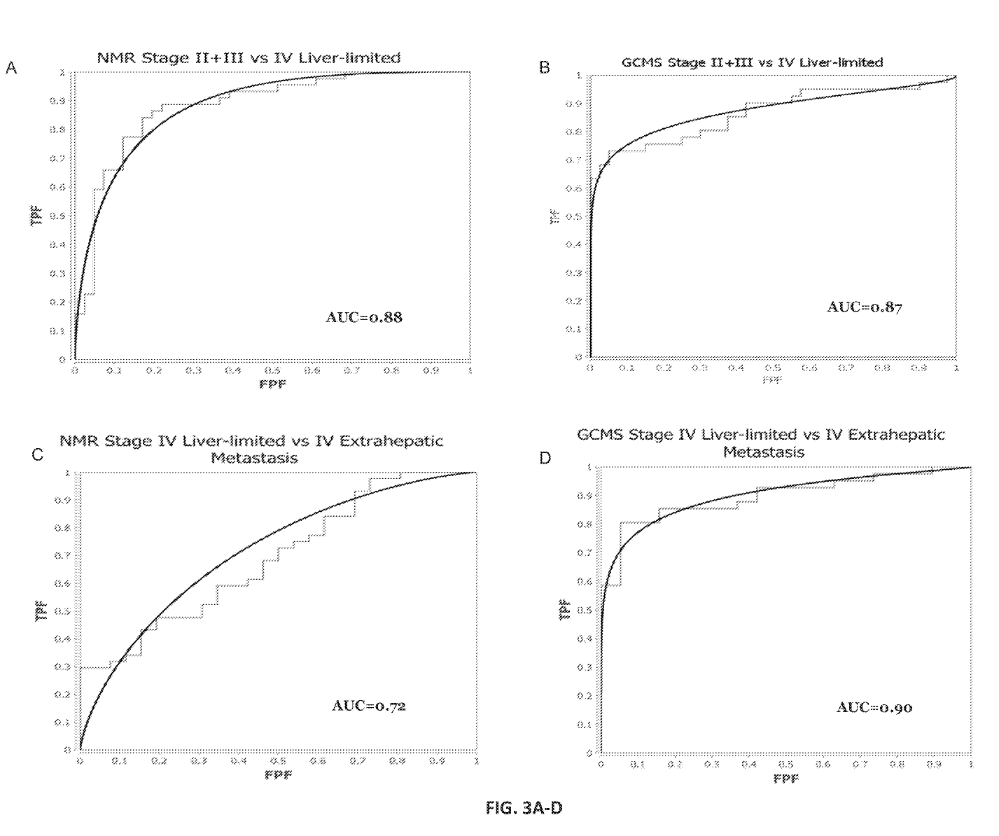

After statistical filtering using a t-test to remove uninformative metabolites, 17 metabolites were included in the regression analysis in ¹H NMR profiling for the comparison of patients with liver-only metastases and patients with extrahepatic metastases. In this instance, orthogonal discriminant analysis did not produce the same strong discriminant components for distinguishing between these groups of patients as was found in the analysis between locoregional CRC and liver-only metastases. In this model, R²Y was only 0.36 and the model was not strongly predictive of metastatic site (Q²Y score=0.13; CV-ANOVA p-value=0.04) (FIG. 2A). Having said this, isoleucine and 2-oxoglutarate were more abundant in sera from patients with extrahepatic metastases, while methionine and fumarate were more abundant in liver-only metastases (FIG. 2C and Tables 2A-D). To refine the model, the stability selection approach was used to find the most important metabolites based on the presence of each metabolite in the list of highly ranked metabolites (explained before). Our final list includes 12 metabolites as the core set and two metabolites as the optional set. Any model with 12 core set and one or both of the optional metabolites have has performed with Q²Y score greater than 0.18 and Area Under the ROC curve of 0.78 or greater (CV-ANOVA p-value=0.007).

Interestingly, GC-MS was more capable of identifying differences between patients with liver-only metastases and extrahepatic metastases. After feature selection of the GC-MS data, 152 components were used for discrimination modeling between these two patient groups, of which 59 were identified as metabolites. The resulting model included metabolites that explained much of the variation in the groups (R²Y score=0.69), and it was predictive (Q²Y score=0.54; CV-ANOVA p-value=4.75×10⁻⁵) (FIG. 2B). FIG. 2D depicts the contributions of each feature to the model. The list was then further purified by the means of VIPs. The resulted in a core list of 25 components and an optional list of 11 components. Models constructed with core set plus any combination of one or more of the optional set will have Q²Y predictive score of above 0.45 and Area Under the ROC curve of greater than 0.92 (CV-ANOVA p-value=4.64×10⁻⁵) Tables 2A-D provides a list of identified metabolites that were seen to be differentially abundant.

Again, to ensure that chemotherapy did not inadvertently affect the inventors' observations, they used the same models in patients who had not been exposed to chemotherapy within 3 months of sample collection. This analysis confirmed that the metabolic profiles continued to be different in the two patient groupings, by ¹H NMR spectroscopy (P=0.69) and by GC-MS (P=3.78×10⁻⁵).

Pathway Analysis.

The inventors were intrigued that the metabolomic profile differed so dramatically in the sera of patients with locoregional disease as compared to liver-only metastases. Further analysis was conducted to glean some understanding of whether this was a reflection of differences in tumor biology, or due to differences in the host response to disease involving different organs, or both. Metabolomic pathway analysis and network analysis were performed using data derived from GC-MS.

Accelerated galactose metabolism was apparent (p value=0.0006 on univariate analysis). The liver is central to galactose metabolism; however, there are no reported alterations in galactose metabolism in tumor cells. Accelerated glutamine and glutamate metabolism was also apparent (p value=0.04 on univariate analysis). Again, the liver is known to actively take up glutamine and convert it to glutamate, making it available for gluconeogenesis or for subsequent conversion to other amino acids. Glutaminolysis is also known to be an important energy source in tumor cells, including in CRC (Turowski et al., 1994; Wasa et al., 1996; Lobo et al., 2000).

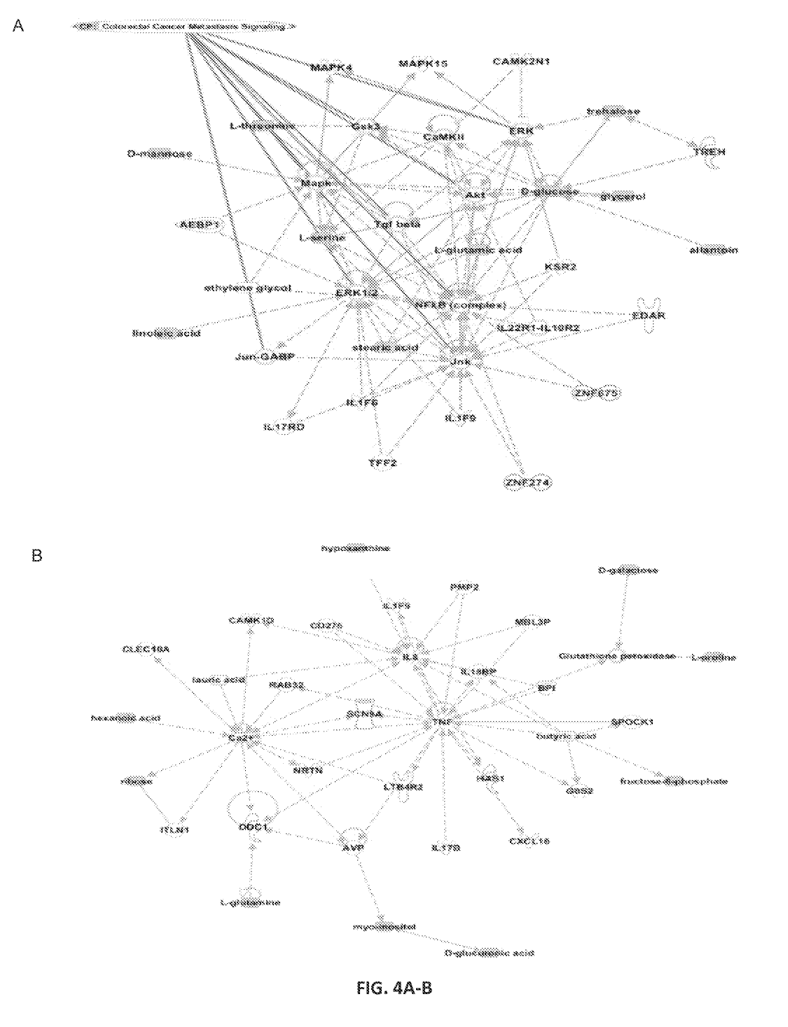

A network analysis was performed, to explore potential upstream altered pathways associated with liver metastases. The IPA network analysis uses information extracted from the literature to extrapolate known signalling and metabolic pathway relationships from the (co-related) metabolites found to be differentially abundant in our experiments. Two networks, representative of observed changes in levels of identified compounds, could be constructed. In the first network, higher levels of NFkB, Mapk and its related CaMKII complex, Jnk and ERK1/2 are predicted to be involved with liver metastasis (FIG. 4A). Interestingly, this combination of signalling complexes and pathways typifies the colorectal cancer metastasis signalling pathway (Sawhney et al., 2006; Wang and Basson, 2011; Sancho et al., 2004; Scartozzi et al., 2007a; Scartozzi et al., 2007b; Messersmith et al., 2005; Vegran et al., 2011; Sakamoto and Maeda, 2010). In this first network, there was also higher activity of several kinases and inflammatory cytokines in the context of liver metastasis. These have not previously been shown to have a direct contribution to metastasis of colorectal cancer. CaMkII, a kinase for several mediators in cell proliferation and apoptosis pathways, is one such molecule. In the second network, a highly-connected web of inflammatory mediators, including TNF, IL-8, and IL-17B, could be visualized (FIG. 4B). IL-17B was recently identified to activate both TNF and NFkB pathways (Iwakura et al., 2011). IL-17B induced expression of TNF and IL-1β results in monocytic chemotaxis (Yamaguchi et al., 2007), a phenomenon which is well described in colorectal liver metastases (Zhou et al., 2010; Giusca et al., 2010).

¹H NMR spectroscopy data were then utilized for pathway analysis. Because a smaller number of metabolites were found to be differentially abundant (compared to GC-MS), it was considered that using these data may not yield a particularly accurate picture of altered metabolic pathways. However, remarkably, the network derived from pathway analysis using ¹H NMR spectroscopy data revealed a role by many of the same signalling molecules and inflammatory mediators demonstrated by analysis of the GC-MS data (data not shown).

The inventors interpreted this analysis to reflect the fact that tumors that metastasize differ biologically from tumors that are confined to the colon. In addition, these data may reflect the response of liver to the local effects of tumor. This pathway analysis therefore supports the hypothesis that the metabolomic profile that distinguishes liver metastases from locoregional CRC reflects elements of a site-specific host response to tumor, as well as changes in tumor biology associated with metastasis.

### Example 3

### Discussion

Presently, preoperative staging for CRC involves radiographic studies such as CT scans to determine extent of disease. Operative findings and pathological examination of the surgical specimen(s) result in a modification of the initially assigned stage. Specifically, the depth of tumor invasion and involvement of lymph nodes are determined. However, in some cases, occult metastatic disease can be missed using contemporary staging methods. Postoperatively, patients are followed closely for local or distant recurrence, in hopes that early detection will hasten treatment before it becomes disseminated. The current guidelines by the American Society of Clinical Oncology suggest annual CT scans for patients eligible for curative surgery (Desch et al., 2005), as well as serum carcinoembryonic antigen (CEA) every 3 months for stage II and III disease for at least 3 years if the patient is a candidate for surgery or chemotherapy for metastatic disease (Locker et al., 2006). This intensive postoperative follow-up is designed to detect metastatic disease that is amenable to resection. For example, limited liver metastases in the absence of extrahepatic metastatic disease may be resected. Biomarkers that facilitate the detection of occult metastatic disease before or after surgery would therefore enhance the staging of CRC patients, potentially impacting on treatment decisions.

Using ¹H NMR spectroscopy and GC-MS, the inventors have demonstrated convincingly using internal validation that the serum metabolomic profile differs in patients with locoregional CRC and metastatic CRC. Moreover, they have observed that there are differences in serum metabolomic profile between patients with metastatic disease that is confined to the liver and extrahepatic metastases. This is a novel finding. External validation will be required to confirm the exact metabolic alterations that occur with each disease state. In addition, more work will be required to determine the sensitivity of the changes. That is, it will be essential to determine the minimal amount of intrahepatic or extrahepatic disease that can be detected by this technique. In order for this biomarker approach to be clinically useful, it must be possible to detect even small, solitary liver metastases, and it must be possible to detect radiographically invisible extrahepatic metastases. These data are promising in this regard, as a large proportion of patients in the liver-only disease group had solitary metastases as small as 14 mm. Finally, the unique and complementary roles of ¹H NMR spectroscopy and GC-MS must be evaluated, for a test that is based on a single analytical modality may be more feasible and cost effective than a test employing two analytical modalities.

Metabolomic biomarkers have numerous advantages over transcriptomic and proteomic biomarkers. First, changes in the metabolome are amplified relative to changes in the transcriptome and proteome (Kell, 2007). Therefore, metabolites may change even when protein levels do not. Second, metabolomic profiling is cheaper and easier than proteomic and transcriptomic profiling. Thus, a test based on metabolomics could be more easily implemented in the clinic. Third, changes in metabolism result in alterations of the abundance of groups of metabolites. Therefore, identification of the patterns of changes in metabolites would provide insight on the functional changes that occur due to any given condition. The metabolomic profile therefore represents a complex biomarker of considerable interest, albeit one that has been studied relatively little.

There have been only four reports so far of serum metabolomic changes associated with CRC, and none have described stage- or organ-specific changes to the metabolomic profile. Qiu et al. compared 64 Chinese patients with CRC to healthy controls; metabolomic profiles were determined by gas chromatography-mass spectrometry (GC-MS) and liquid chromatography-mass spectrometry (LC-MS) (Qiu et al., 2009; Kondo et al., 2011). The metabolomic profiles in CRC patients (including 8 patients with stage IV CRC) were distinct from those of healthy controls. Interestingly, there were a number of metabolites that were differentially abundant in all stages of disease. This study demonstrated the feasibility of using metabolomics to diagnose CRC. Kondo et al. (2011) similarly used GC-MS to demonstrate that serum fatty acid composition differed in a small cohort of Japanese CRC patients compared to healthy controls (Kondo et al., 2011). Since only 20 patients were examined, it was not feasible to evaluate differences in subgroups. Ludwig et al. used NMR spectroscopy to delineate the metabolomic signature of 38 patients with various stages of CRC (including 20 patients with stage IV disease), and identified a typical Warburg signature in association with CRC (Ludwig et al., 2009). The only group so far to specifically study patients with metastatic CRC did not evaluate site of disease as a contributing factor in metabolomic profile (Bertini et al., 2012). Moreover, their study population consisted of patients who had been heavily pretreated with multiple cytotoxic chemotherapy regimens. Therefore, the metabolomic profile derived may not be entirely representative of metastatic CRC in general. Interestingly, there were differences in abundance of a number of metabolites between patients who had short survivals and longer survivals. The findings in each of these series will require validation, and further work will be required to evaluate differences in findings in populations from different countries that may occur due to differences in dietary, environmental and genetic factors. Moreover, additional research will be required to identify disease factors which modify the metabolomic signature, including tumor biology, stage and the host response.

One factor that must be further evaluated in the context of this series is the effect of chemotherapy. Patients with metastatic disease were more frequently exposed to chemotherapy within 3 months of sample collection, and it is possible that this influenced the results reported here to some degree. Having said this, there are two lines of evidence that chemotherapy exposure did not have a significant effect. First, regression analysis demonstrated no statistically significant effect on the metabolomic profile. This may be because the time between the last dose of chemotherapy and the date of sample collection was sufficient to “wash out” any residual metabolic effects of these drugs. Second, the inventors determined that the models derived were unchanged even in individuals who had not received chemotherapy. Ultimately, it will be important to validate our findings in a larger cohort that was not exposed to chemotherapy prior to sample collection.

The finding that metabolomic profile changes with site of disease was surprising and intriguing. The question is whether changes in the circulating metabolites reflect differences in tumor biology or alterations in the host response to tumor, or a combination of both. The host response may change with metastasis because metastatic disease is, by definition, biologically distinct from a cancer that remains confined in the tissue of origin; and more aggressive tumors may incite a more (or less) exuberant response by the host. The response of the host may also differ because of the local effects of tumor. For example, a tumor may have numerous paracrine effects on the surrounding microenvironment, and the metabolic or inflammatory response of surrounding normal tissues may differ between colon, liver and other metastatic sites. The pathway analysis is meant to be hypothesis generating, and this analysis suggested that tumor biology and the host response may both be contributing to the changes in serum metabolomic profile seen with site of disease. Further experimentation on the contributions of various tissues to the circulating metabolome will be required to delineate the relative effects of tumor and host.

In addition to the limitations described above, it is possible that the performance of these metabolomic tests is the result of over-fitting. On the other hand, the generated models demonstrate acceptable and often excellent goodness of fit, as well as satisfactory goodness of prediction for human sample type metabolomic studies. The inventors will further validate the models with an independent patient cohort, thereby confirming that these metabolites are useful in a clinical setting.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

### V. REFERENCES

The following references, to the extent that they provide exemplary procedural or other details supplementary to those set forth herein, are specifically incorporated herein by reference:
Bathe et al., BMC Cancer, 9:156, 2009.
Bathe et al., Cancer Epidemiol. Biomarkers Prev., 20(1):140, 2011.
Bertini et al., Cancer Res., 72(1):356-364, 2012.
Bligh and Dyer, Ca. J. Biochem. Physiol., 37(8):911-917, 1959.
Desch et al., J. Clin. Oncol., 23(33):8512-8519, 2005.
Giusca et al., Romanian J. Morph. Embryol., 51(1):73-79, 2010.
Hummel et al., In: The Golm Metabolome Database: a database for GC-MS based metabolite profiling Metabolomics, Nielsen and Jewett (Eds.), Springer Berlin/Heidelberg, 18:75-95, 2007.
Iwakura et al., Immunity, 34(2):149-162, 2011.
Kell, Expert Rev. Molec. Diagnos., 7(4):329, 2007.
Kondo et al., Biomark. Med., 5(4):451-460, 2011.
Lobo et al., Biochem. J., 348(2):257-261, 2000.
Locker et al., J. Clin. Oncol., 24(33):5313-5327, 2006.
Ludwig et al., Magnetic Reson. Chem., 47(1):568-73, 2009.
Messersmith et al., Cancer Biol., Therapy, 4(12):1381-1386, 2005.
Pawlik et al., Ann. Surg., 241(5):715, 2005.
Pawlik et al., Oncologist, 13(1):51-64, 2008.
Qiu et al., J. Proteome Res., 8(10):4844, 2009.
Sakamoto and Maeda, Expert Opin. Therap, Targets, 14(6):593-601, 2010.
Sancho et al., Ann. Rev. Cell Develop. Biol., 20:695-723, 2004.
Sawhney et al., J. Biol. Chem., 281(13):8497-8510, 2006.
Scartozzi et al., Br. J. Cancer, 97(1):92-97, 2007a.
Scartozzi et al., J. Clin. Oncol., 25(25):3930-3935, 2007b.
Shah et al., J. Am. Coll. Surg., 205(5):676, 2007.
Stein, J. Amer. Soc. Mass Spectro., 6(8):644-655, 1995.
Turowski et al., Cancer Res., 54(22):5974-5980, 1994.
Vegran et al., Cancer Res., 71(7):2550-2560, 2011.
Wang and Basson, Amer. J. Physiol. Cell Physiol., 300(3):C657-670, 2011.
Wasa et al., Ann., Surgery, 224(2):189-197, 1996.
Weljie et al., Analytical Chemistry, 78(13):4430, 2006.
Weljie et al., J. Proteome Res., 6(9):3456-3464, 2007.
Wishart et al., Nucleic Acids Res., 37:D603-610, 2009.
Xia et al., Nucleic Acids Res., 37(2):W652-W660, 2009.
Yamaguchi et al., J. Immunol., 179(10):7128-7136, 2007.
Zhou et al., J. Translat. Med., 8:13, 2010.
Bylesjö et al., J Chemometrics, 20:341-51, 2006.
Roe and Metz, Academic Radiol, 4:298-303, 1997.
Wehrens et al., Analytica chimica acta, 705(1):15-23, 2011.
R Development Core Team, R Foundation for Statistical Computing, Vienna, Austria, 2013.

1. A method of characterizing metastatic colorectal cancer comprising:
(a) obtaining a mammalian biological sample;
(b) analyzing the biological sample from the mammal with gas chromatography-mass spectrometry to determine the level(s) of one or more core biomarkers set forth in Table 2B and Table 2D; and
(c) comparing the level(s) of the one or more core biomarkers in the sample to metastatic and/or normal reference levels of the one or more core biomarkers in order to assess the characterize metastatic disease in the mammal.

2. The method of claim 1, wherein step (b) comprises analyzing the levels of two, three, four, five, six, seven, eight, nine, ten, eleven, twelve, thirteen, fourteen, fifteen, sixteen, seventeen, eighteen, nineteen, twenty, twenty-five, thirty, thirty-five, forty, fifty or all of the core biomarkers in Table 2B and Table 2D.

3. The method of claim 1, wherein the biological sample is a body fluid.

4. The method of claim 3, wherein the body fluid is a urine sample, a blood sample, a serum sample or a plasma sample.

5. The method of claim 1, wherein analyzing comprises determining one or more core markers in Table 2B.

6. The method of claim 5, wherein analyzing comprises determining all the core markers in Table 2B.

7. The method of claim 1, wherein analyzing comprises determining one or more core markers in Table 2D.

8. The method of claim 7, wherein analyzing comprises determining all the core markers in Table 2D.

9. The method of claim 1, wherein said mammal is a human.

10. The method of claim 1, wherein characterizing distinguishes liver-only colorectal metastasis from locoregional colorectal cancer or extrahepatic colorectal metastasis.

11. A kit for characterizing metastatic colorectal cancer in a mammal, the kit comprising reagents suitable for determining levels of a plurality of core biomarkers in a test sample using gas chromatography-mass spectrometry, wherein the plurality of core biomarkers comprises two or more of the core biomarkers in Table 2B and Table 2D.

12-15. (canceled)

16. A method of staging metastatic colorectal cancer comprising:
(a) obtaining a mammalian biological sample;
(b) analyzing the biological sample from the mammal using gas chromatograph-mass spectrometry to determine the level(s) of one or more core biomarkers set forth in Table 2B and Table 2D; and
(c) comparing the level(s) of the one or more core biomarkers in the sample to metastatic and/or normal reference levels of the one or more core biomarkers in order to stage disease in the mammal.

17. A method of determining an appropriate therapy for colorectal cancer comprising:
(a) obtaining a mammalian biological sample;
(b) analyzing the biological sample from the mammal using gas chromatograph-mass spectrometry to determine the level(s) of one or more core biomarkers set forth in Table 2B and Table 2D; and
(c) comparing the level(s) of the one or more core biomarkers in the sample to metastatic and/or normal reference levels of the one or more core biomarkers in order to assess the extent of metastatic disease in the mammal, thereby indicating the appropriate type of therapy.

18-20. (canceled)

21. A method of characterizing metastatic colorectal cancer comprising:
(a) obtaining a mammalian biological sample;
(b) analyzing the biological sample from the mammal with ¹H-NMR spectroscopy to determine the level(s) of one or more core biomarkers set forth in Table 2A and Table 2C; and
(c) comparing the level(s) of the one or more core biomarkers in the sample to metastatic and/or normal reference levels of the one or more core biomarkers in order to assess the characterize metastatic disease in the mammal.

22. The method of claim 21, wherein step (b) comprises analyzing the levels of two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six or all of the core biomarkers in Table 2A and Table 2C.

23. The method of claim 21, wherein the biological sample is a body fluid.

24. The method of claim 23, wherein the body fluid is a urine sample, a blood sample, a serum sample or a plasma sample.

25. The method of claim 21, wherein analyzing comprises determining one or more core markers in Table 2A.

26. The method of claim 25, wherein analyzing comprises determining all the core markers in Table 2A.

27. The method of claim 21, wherein analyzing comprises determining one or more core markers in Table 2C.

28. The method of claim 27, wherein analyzing comprises determining all the core markers in Table 2C.

29. The method of claim 21, wherein said mammal is a human.

30. The method of claim 21, wherein characterizing distinguishes liver-only colorectal metastasis from locoregional colorectal cancer or extrahepatic colorectal metastasis.

31. A kit for characterizing metastatic colorectal cancer in a mammal, the kit comprising reagents suitable for determining levels of a plurality of core biomarkers in a test sample using ¹H-NMR spectroscopy, wherein the plurality of core biomarkers comprises two or more of the core biomarkers in Table 2A and Table 2C.

32-35. (canceled)

36. A method of staging metastatic colorectal cancer comprising:
(a) obtaining a mammalian biological sample;
(b) analyzing the biological sample from the mammal using ¹H-NMR spectroscopy to determine the level(s) of one or more core biomarkers set forth in Table 2A and Table 2C; and
(c) comparing the level(s) of the one or more core biomarkers in the sample to metastatic and/or normal reference levels of the one or more core biomarkers in order to stage disease in the mammal.

37. A method of determining an appropriate therapy for colorectal cancer comprising:
(a) obtaining a mammalian biological sample;
(b) analyzing the biological sample from the mammal using ¹H-NMR spectroscopy to determine the level(s) of one or more core biomarkers set forth in Table 2A and Table 2C; and
(c) comparing the level(s) of the one or more core biomarkers in the sample to metastatic and/or normal reference levels of the one or more core biomarkers in order to assess the extent of metastatic disease in the mammal, thereby indicating the appropriate type of therapy.

38-40. (canceled)

41. The method of claim 1, further comprising analyzing the biological sample from the mammal using ¹H-NMR spectroscopy to determine the level(s) of one or more optional biomarkers set forth in Table 2A and Table 2C.

42. The method of claim 21, further comprising analyzing the biological sample from the mammal using gas chromatography to determine the level(s) of one or more optional biomarkers set forth in Table 2B and Table 2D.

43-44. (canceled)
